(19) United States
(12) Patent Application Publication
(10) Pub. No.: US 2026/0314956 A1
(43) Pub. Date: Oct. 15, 2026

(54) Mobility Support Apparatus With Integrated Biomechanical Monitoring and Rehabilitation Analytics

(71) Applicant: Lisa Marie OKeefe, Binghamton, NY (US)

(72) Inventor: Lisa Marie OKeefe, Binghamton, NY (US)

(21) Appl. No.: 19/573,453

(22) Filed: Mar. 20, 2026

Related U.S. Application Data
(60) Provisional application No. 63/788,955, filed on Apr. 15, 2025.

(51) Int. Cl. A61B 5/11; A45B 3/08; A61B 5/00

(52) U.S. Cl. CPC A61B 5/112; A45B 3/08; A61B 5/0004; A61B 5/7275; A61B 5/7445; A61B 5/7455; A61B 2505/09

(57) ABSTRACT

A mobility support apparatus configured to assist a user during ambulation while simultaneously monitoring biomechanical interaction between the user and the apparatus. The apparatus includes one or more sensors configured to measure force, motion, orientation, or positional characteristics associated with use of the mobility support apparatus. Sensor data is processed by an internal processing unit to generate analytics related to gait characteristics, rehabilitation progress, mobility performance, or fall risk. The apparatus may further provide feedback to the user through visual, tactile, or auditory indicators and may communicate wirelessly with external computing devices for data storage, analysis, or remote monitoring. The apparatus may include configurable components, moisture-resistant housings, and modular sensor placement within structural elements of the mobility support device.

### FIELD OF THE INVENTION

The present invention relates generally to mobility assistance devices, and more particularly to mobility support apparatuses incorporating integrated sensor systems configured to monitor gait, balance, rehabilitation progress, and biomechanical interaction between a user and a mobility support device.

### BACKGROUND

Mobility support devices such as canes, walking poles, walkers, and other support structures are commonly used by individuals experiencing mobility limitations due to aging, injury, surgery, neurological disorders, or musculoskeletal conditions.

Traditional mobility aids provide structural assistance but lack the capability to measure or analyze how the user interacts with the device during walking or rehabilitation. Healthcare professionals often rely on periodic observation rather than continuous measurement of gait patterns or weight distribution.

There is therefore a need for a mobility support device capable of monitoring biomechanical interaction between the user and the device and generating analytics related to gait characteristics, rehabilitation progress, balance, and fall risk.

### SUMMARY OF THE INVENTION

The present invention provides a mobility support apparatus incorporating one or more sensors configured to measure interaction between the user and the device.

The apparatus may detect parameters including but not limited to:
force or pressure applied to the device
motion or acceleration
angular orientation or tilt
distance or displacement
gait rhythm or stability patterns

Sensor data may be processed by an internal microcontroller or processor and may be transmitted to external devices for analysis or monitoring.

The apparatus may further provide real-time feedback to the user through visual indicators, tactile feedback, audio alerts, or mobile applications.

The system may assist with monitoring rehabilitation progress, identifying gait irregularities, detecting fall events, or providing mobility analytics over time.

### BRIEF DESCRIPTION OF THE DRAWINGS

FIG. 1 is a front view of a mobility support apparatus.

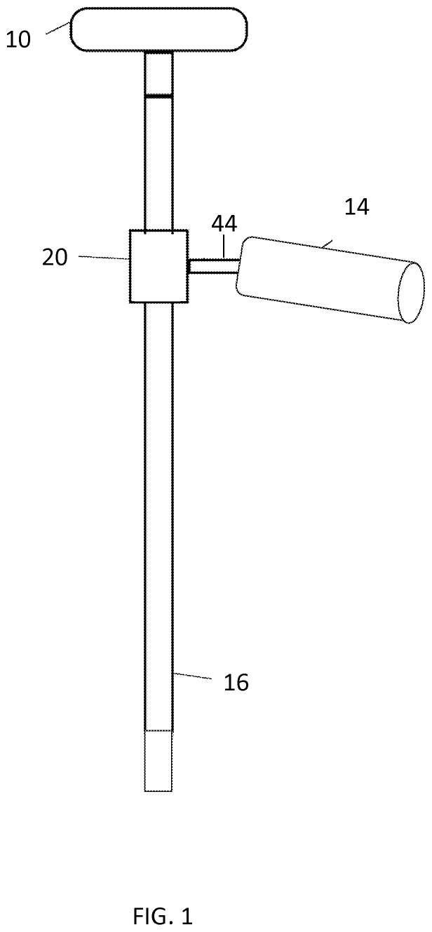

FIG. 2 is a diagram illustrating internal components of a sensor module.

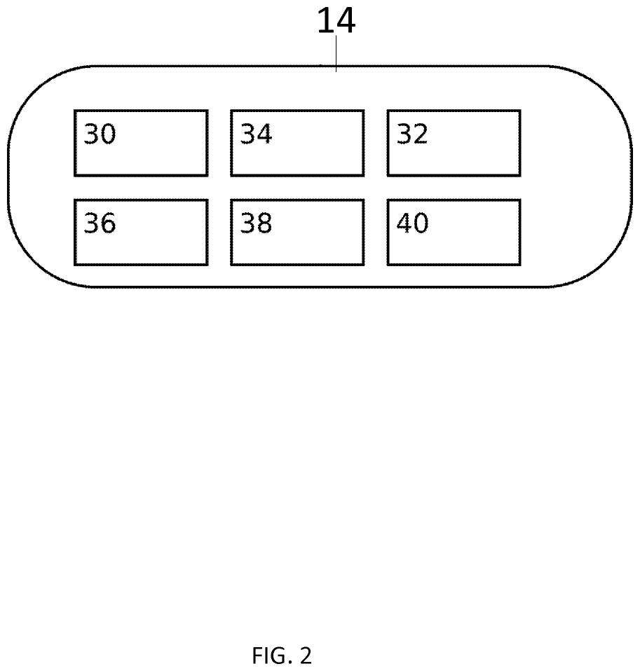

FIG. 3 is a side view of the sensor module connected to the support structure.

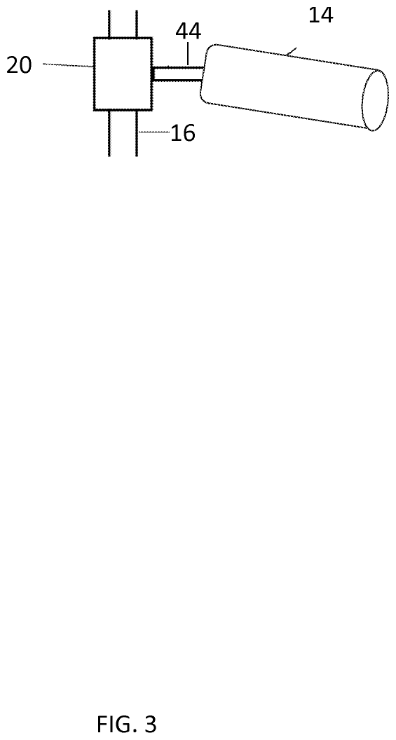

FIG. 4 is a side view of the apparatus showing sensor placement.

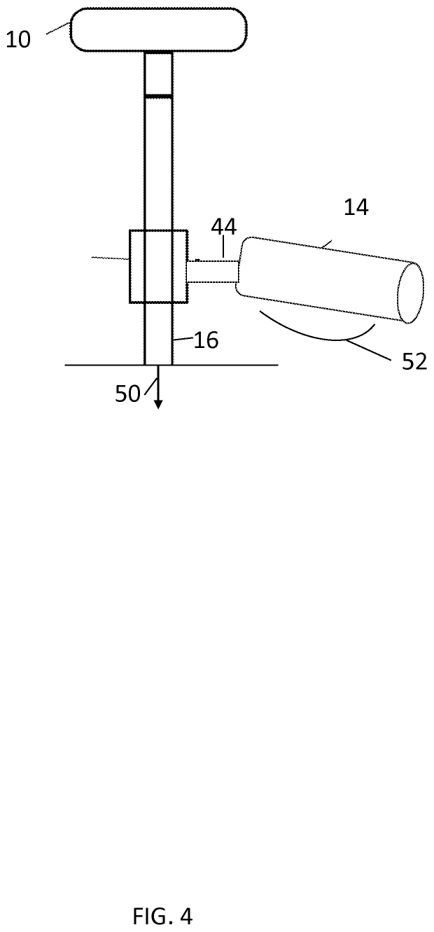

FIG. 5 is a diagram illustrating communication between the apparatus and an external mobile device.

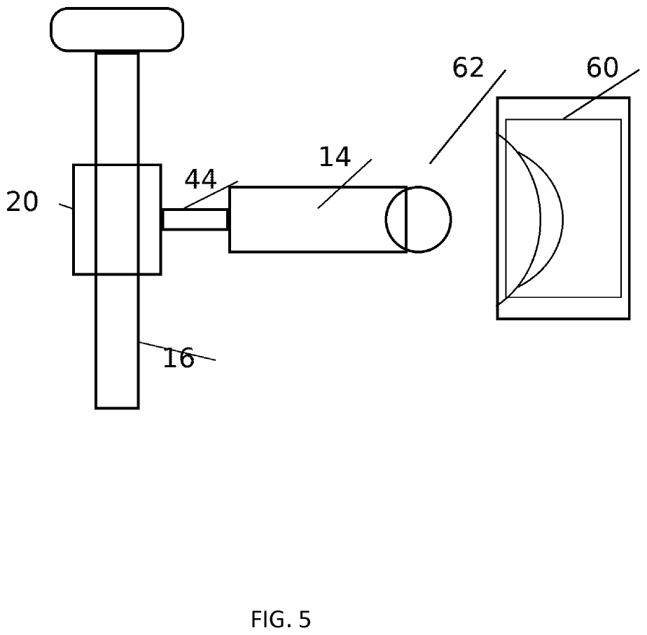

FIG. 6 is a perspective view of the sensor module with feedback indicators.

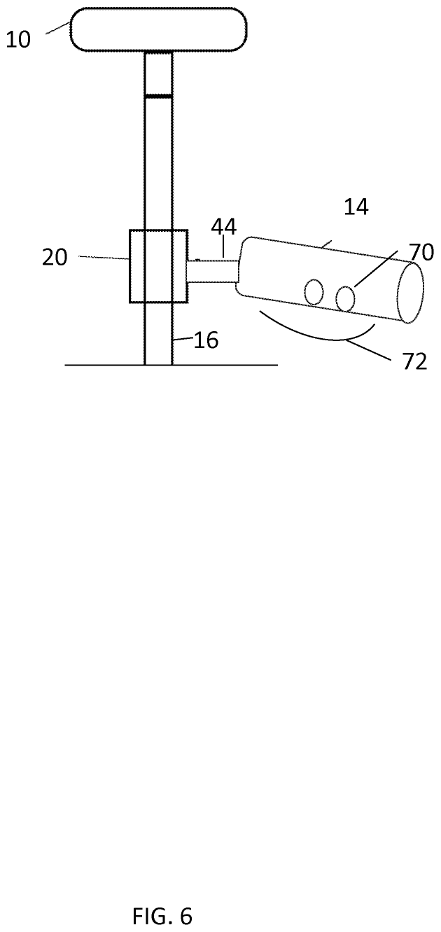

FIG. 7 is a view illustrating feedback signals generated by the apparatus.

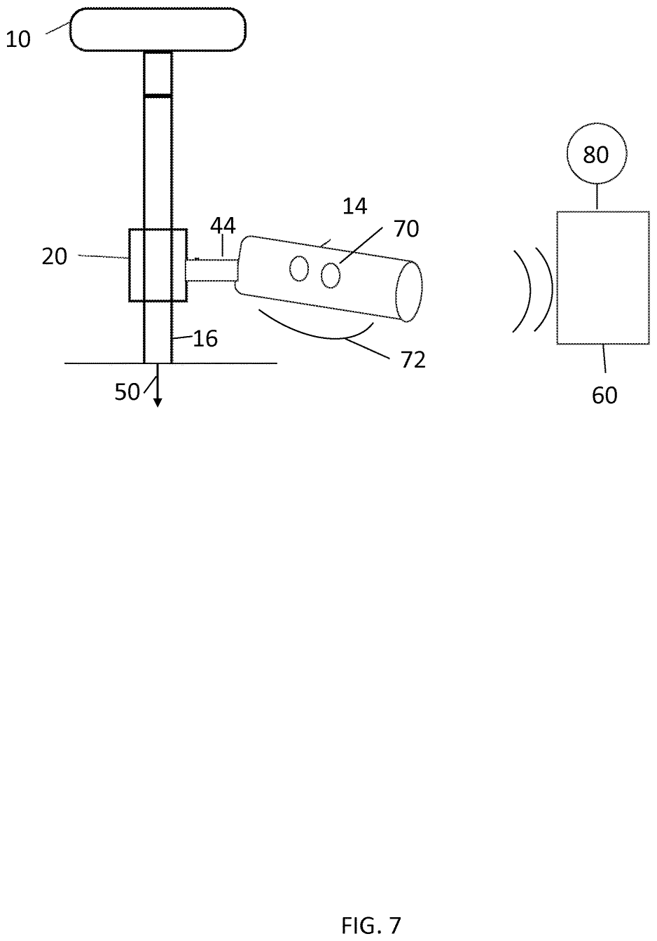

FIG. 8 is a schematic diagram of the sensor, processing, communication, and feedback system.

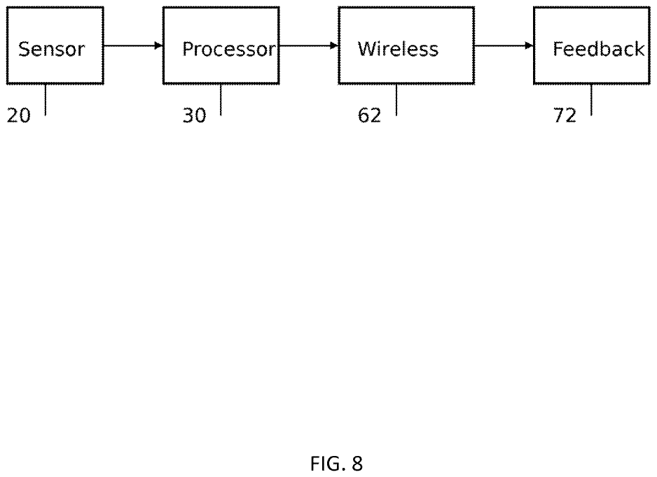

### DETAILED DESCRIPTION

### Device Structure

The mobility support apparatus may include:
a structural support member configured to support a user during ambulation
a handle or grip portion
a shaft or structural body
a base or ground-contact tip
an electronics housing integrated within the apparatus

The apparatus may be configured as:
a cane
a walking pole
a walker support element
a rehabilitation support device
or other handheld mobility assistance structure.

Referring to the drawings, the apparatus includes a handle (10), a shaft (16), and a sensor module (14) connected via a connector (44) and bracket (20). A base or ground-contact tip (50) supports the apparatus.

### Sensor System

One or more sensors may be incorporated into the apparatus including but not limited to:
pressure sensors
force sensors⋅load cells⋅accelerometers⋅gyroscopes⋅magnetometers⋅optical sensors⋅distance sensors

Sensors may be positioned in one or more locations including:
within the handle
within the shaft
within the mid-shaft region
near the base tip
or within internal structural components.

The sensors may operate independently or in combination to measure biomechanical interaction between the user and the mobility support apparatus.

In some embodiments, one or more force or pressure sensors may be positioned within a mid-shaft region of the apparatus. In other embodiments, sensors may be positioned at any location along the structural support member, including but not limited to the handle, shaft, base, or internal structural regions, to measure load distribution, balance characteristics, or user interaction.

Such sensor placement enables measurement of load distribution along the structural support member and may be used to detect gait imbalance, uneven weight loading, or changes in rehabilitation progress.

Sensor placement within or along internal or external structural elements of the apparatus may vary in position, orientation, quantity, and configuration and is not limited to any specific region of the apparatus.

The sensor module (14) may include internal components such as a processor (30), control circuitry or memory (34), and a wireless communication module (32). Additional sensors may include motion sensors (36), pressure sensors (38), and detection elements (40).

In some embodiments, the apparatus may include a contact surface or sensor interface (52) positioned along a lower portion of the sensor module or structural member, configured to detect pressure, load distribution, or user interaction with the apparatus.

### Communication and Feedback

The apparatus may communicate with an external device such as a mobile device (60) via a wireless communication signal (62). Feedback may be provided through a visual indicator (70) or feedback output (72). In some embodiments, data may be transmitted to a remote system or cloud platform (80).

### Processing System

The apparatus may include a microcontroller or processor configured to:
receive sensor data
analyze biomechanical interaction patterns
generate mobility analytics
detect abnormal movement patterns or falls
control feedback systems.

Algorithms may evaluate factors including:
weight distribution
stride timing
gait symmetry
tilt patterns
sudden acceleration events.

### Feedback System

The apparatus may provide feedback to the user through:
LED visual indicators
vibration motors
audio alerts
visual displays
connected mobile applications.

Feedback may indicate:
excessive weight loading
balance instability
rehabilitation progress
fall detection alerts.

Feedback may be provided through a visual indicator (70) or feedback output (72).

### Communication System

The apparatus may include a communication interface configured to transmit data to external devices including:
smartphones
tablets
computers
healthcare monitoring platforms.

Communication protocols may include:
Bluetooth
Wi-Fi
cellular communication
wired interfaces.

The apparatus may communicate with an external mobile device (60) via a wireless communication signal (62) and may interface with a remote system (80).

### Power System

The apparatus may include one or more power sources, including but not limited to:
rechargeable battery
removable battery⋅inductive charging system⋅wired charging interfaces such as USB-C or magnetic connectors⋅energy-harvesting systems including solar, kinetic, or other environmental energy sources

The power system may be configured to support continuous operation, intermittent use, or automatic power management based on device activity.

The foregoing description is illustrative and not limiting, and variations in form, configuration, and implementation may be made without departing from the scope of the invention.

1. A mobility support apparatus comprising:
a structural support member configured to assist a user during ambulation;
one or more sensors configured to measure biomechanical interaction between the user and the apparatus;
a processing unit configured to analyze data generated by the sensors to determine mobility characteristics of the user; and
an output interface configured to provide feedback or communicate the mobility characteristics.

2. The apparatus of claim 1, wherein the sensors comprise one or more force or pressure sensors configured to measure load applied to the apparatus.

3. The apparatus of claim 1 wherein the sensors comprise motion sensors configured to detect acceleration, orientation, or tilt.

4. The apparatus of claim 1, wherein at least one sensor is positioned along a portion of the structural support member between a handle region and a base region, including a mid-shaft region, and is configured to measure load distribution, balance characteristics, or user interaction independent of grip pressure.

5. The apparatus of claim 1 wherein the apparatus generates gait metrics based on sensor data.

6. The apparatus of claim 1 wherein the output interface comprises visual indicators including light-emitting diodes.

7. The apparatus of claim 1 wherein the output interface comprises tactile feedback.

8. The apparatus of claim 1 wherein the apparatus communicates mobility data to an external computing device.

9. The apparatus of claim 1 wherein the apparatus detects fall events or fall-risk conditions.

10. The apparatus of claim 1 wherein the structural support member comprises a cane, walking pole, walker component, or rehabilitation support structure.

11. A method of monitoring mobility using a mobility support device comprising:
collecting sensor data representing biomechanical interaction between a user and a mobility support device;
analyzing the sensor data to determine gait characteristics; and
generating feedback or mobility analytics based on the analysis.

12. The method of claim 11 wherein the feedback indicates rehabilitation progress or fall risk.

13. A mobility monitoring system comprising:
a mobility support device containing sensors;
a mobile computing device configured to receive sensor data; and
software configured to analyze mobility characteristics and generate rehabilitation analytics.

14. A mobility monitoring system comprising:
a mobility support apparatus including one or more sensors configured to measure biomechanical interaction between a user and the apparatus;
a processing module configured to generate mobility data based on the measured interaction; and
an analytics module configured to analyze the mobility data to produce one or more rehabilitation metrics including gait symmetry, weight distribution, balance stability, or mobility progression over time.

15. The system of claim 14 wherein the analytics module generates visual reports representing mobility trends across multiple walking sessions.

16. The system of claim 14 wherein the analytics module compares current mobility data with historical data to identify rehabilitation progress or decline.

17. The system of claim 14 wherein the analytics module is implemented on a mobile application, cloud server, or external computing device.

18. The system of claim 14 wherein the analytics module generates alerts when sensor data indicates abnormal mobility patterns or elevated fall risk.

19. The system of claim 14 wherein mobility metrics are transmitted to caregivers, therapists, or healthcare providers.
